(19) United States
(12) Patent Application Publication
(10) Pub. No.: US 2003/0096387 A1
(43) Pub. Date: May 22, 2003

(54) Peptidyl-prolyl cis-trans isomerase inhibitors and uses therefor

(75) Inventors: Joseph P. Noel, San Diego, CA (US); Tony R. Hunter, Del Mar, CA (US)

Correspondence Address: FOLEY & LARDNER, P.O. BOX 80278, SAN DIEGO, CA, 92138-0278, US

(73) Assignee: The Salk institute for Biological Studies

(21) Appl. No.: 10/282,588

(22) Filed: Oct. 28, 2002

Related U.S. Application Data
(63) Continuation of application No. 09/094,436, filed on Jun. 9, 1998.

(51) Int. Cl. C12N 9/00; C12N 9/99

(52) U.S. Cl. 435/183; 435/184

(57) ABSTRACT

The invention relates to inhibitors of PPIase activity and pharmaceutical compositions containing such inhibitors. Such compounds are useful for treatment of disorders characterized by inappropriate cell proliferation. In particular the compounds disclosed herein inhibit the activity of members of the Pin1/parvulin class of PPIases. These compounds have been designed based on the high resolution X-Ray derived crystal structure of the human enzyme Pin1.

### FIELD OF THE INVENTION

The present invention relates to inhibitors of peptidyl-prolyl cis-trans isomerases which are useful for the therapeutic treatment of various disorders, pharmaceutical compositions comprising these inhibitors and methods of using them. The invention further describes the three-dimensional structures of an exemplary peptidyl-prolyl cis-trans isomerase, Pin1, complexed with a substrate mimic or complexed with an inhibitor of Pin1 activity. Methods of rational drug design using these three-dimensional structures to design new inhibitors are also contemplated by the present invention.

### BACKGROUND OF THE INVENTION

The process of designing potent and specific inhibitors has improved with the arrival of techniques for determining the three-dimensional structure of the enzyme to be inhibited. Usually a three-dimentional model of an enzyme is produced by the creation of a crystalline form of the purified enzyme which is then subjected to X-ray diffraction and analysis. While such procedures provide certain valuable information that can be used to design inhibitors, they suffer from a lack of knowledge about the amino acid residues critical for interaction with a substrate or a substrate mimic. In order to address these limitations, enzymes have more recently been co-crystalized with substrates, substrate mimics or known inhibitors of the enzyme's activity, thereby allowing the important interactions to be determined (see, for example, Mohammadi, et al, Science 276:955-960, 1997; Lee, et al, Biochemistry 36:13180-13186, 1997; Brzozowski, et al, Nature 389:753-758, 1997).

The peptidyl-prolyl cis-trans isomerases (PPIases), or rotamases, are a family of enzymes important in protein folding, assembly and transport. They act as catalysts to promote isomerization about the peptidyl-prolyl bond, which can have profound effects on protein function.

PPIases are divided into three classes, cyclophilins, FK-506 binding proteins (FKBPs) and the Pin1/parvulin class. While cycloohilins and FKBPs are distinguished by their ability to bind immunosuppressant molecules cyclosporin and FK-506, respectively, the Pin1/parvulin class binds neither of these immunosupressants and is structurally unrelated to the other two classes. Known members of the Pin1/parvulin class include Pins 1-3 (Lu, et al, Nature 380:544-547, 1996), Pin-L (Campbell, et al, Genomics 44:157-162, 1997), parvulin (Rahfeld, et al, FEBS Letts 352:180-184, 1994), dodo (Maleszka, et al, Proc Natl Acad Sci USA 93:447-451, 1996) and Ess1/Pft1 (Hanes, et al, Yeast 5:55-72, 1989; and Hani, et al, FEBS Letts 365:198-202, 1995).

Recent research suggests that members of the Pin1/parvulin class are essential modulators of the cell cycle, and mitosis in particular. Lu, et al, Nature 380:544-547, 1996 (incorporated by reference herein) reports that depletion of Pin1/Ess1 in yeast or human cells induces mitotic arrest followed by apoptosis, indicating that enzymes in this class serve an essential function in cell division and proliferation.

The design of new, highly specific antimitotic agents represents an important need in the pharmaceutical industry. Such agents can serve as effective chemotherapeutic agents for the treatment of a variety of disorders characterized by inappropriate cell proliferation, including cancer and infectious diseases. The invention disclosed herein addresses this and related needs, as will become apparent upon review of the specification and appended claims.

### BRIEF DESCRIPTION OF THE INVENTION

In accordance with the present invention, there are provided methods and compounds for inhibiting peptidyl-prolyl cis-trans isomerases, also called PPIases. In particular, invention compounds inhibit the activity of members of the Pin1/parvulin class of PPIases, which assume an important function in the cell cycle, particularly with respect to mitosis. The compounds of the invention can, therefore, be used in pharmaceutical compositions for the treatment of disorders characterized by inappropriate cell proliferation (e.g., cancer) as well as infectious diseases (e.g., bacterial infections, fungal infections), and the like.

Also provided herein is the three-dimensional crystalline structure of Pin1, a specific peptidyl-prolyl cis-trans isomerase, and methods for utilizing this structure to design specific inhibitors of Pin1 activity as well as inhibitors of other members of the parvulin subfamily of PPIases.

### BRIEF DESCRIPTION OF THE FIGURES

FIG. 1 is a ribbon representation of crystalline Pin1.

FIG. 2 is a closeup of the active site of crystalline Pin1, bound to the substrate mimic H₃N⁺-AlaPro-COO⁻.

### DETAILED DESCRIPTION OF THE INVENTION

In accordance with the present invention, there are provided inhibitors of PPIases, in particular members of the Pin1/parvulin subfamily of PPIases. In general, invention inhibitors have the structure I as follows:

A-X-R (I)

wherein A is a radical which mimics the steric and electronic properties of a phosphoserine and/or phosphothreonine residue, X is a spacer, and R is a ring structure which is at least as hydrophobic as a pyrrolidine ring substituted with a hydrophilic moiety.

As readily recognized by those of skill in the art, a variety of spacers can be employed in the practice of the present invention, For example, spacer X can be selected from

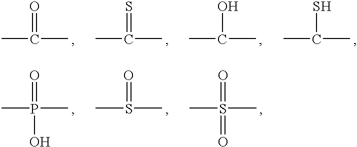

and the like.

R can be any one of a variety of cyclic systems which mimic the steric and electronic properties of a prolyl radical. Thus, R can be, for example, cycloalkyl, substituted cycloalkyl, heterocyclic, substituted heterocyclic, aryl, substituted aryl, heteroaryl, substituted heteroaryl, and the like.

Radical A of structure I can similarly be selected from a variety of radicals which mimic the steric and electronic properties of a phosphoserine and/or phosphothreonine residue. Examples of suitable radicals having these properties include radicals II, III, or IV as follows:

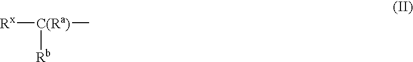

wherein:

R^{x} is an organic radical having a molecular weight no greater than about 250,

R^{a} is H, halo or lower alkyl, and

R^{b} is —(CR^{c}₂)₁₋₄—CHₘY₃₋ₘ,

wherein:

each Y is independently selected from —OR^{d}, —COOR^{c}, —CF₃, —P(O)(OR^{c})₂, —OP(O)(OR^{c})₂, —NH—P(O)(OR^{c})₂, or NH—CH(CF₃)₂ wherein each R^{c} is independently H or lower alkyl, each R^{d} is independently H, lower alkyl, alkylcarbonyl, and m=1 or 2, or

R^{z}—NH— (III)

wherein R^{z} is alkyl, substituted alkyl cycloalkyl, substituted cycloalkyl, cycloalkenyl, substituted cycloalkenyl, cycloalkadienyl, substituted cycloalkadienyl, heterocyclic, substituted heterocyclic, mono- or poly-unsaturated heterocyclic or substituted mono- or poly-unsaturated heterocyclic, aryl, substituted aryl, heteroaryl, substituted heteroaryl, and the like, or

R^{b}-substituted Cy(het)- (IV)

wherein Cy(het) is a 5, 6 or 7-membered heterocyclic ring wherein the heterocyclic atom thereof is linked to X of structure I, and R^{b} is as defined above.

In moiety II above, the organic radical, RX, can be any one of a variety of substituents, such as, for example, alkyl, substituted alkyl, cycloalkyl or substituted cycloalkyl, cycloalkenyl or substituted cycloalkenyl, cycloalkadienyl or substituted cycloalkadienyl, heterocyclic or substituted heterocyclic, mono- or poly-unsaturated heterocyclic or substituted mono- or poly-unsaturated heterocyclic, aryl or substituted aryl, heteroaryl or substituted heteroaryl, or:

R^{y}-Q-

wherein:

R^{y} is alkyl, substituted alkyl, cycloalkyl, substituted cycloalkyl, cycloalkenyl, substituted cycloalkenyl, cycloalkadienyl, substituted cycloalkadienyl, heterocyclic, substituted heterocyclic, mono- or poly-unsaturated heterocyclic or substituted mono- or poly-unsaturated heterocyclic, aryl, substituted aryl, heteroaryl, or substituted heteroaryl, and

Q is —C(O)—NH—, —C(O)—O—, —C(O)—, or —O—.

In particular, R^{x} can be an amino acid residue (e.g., a leucinyl moiety, a prolyl moiety, and the like), as well as:

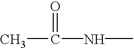

when A is structure II above.

In moiety III above, specific examples of R^{z} include:

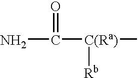

wherein R^{a} and R^{b} are as defined above, or moiety III can be:

(R^{b}′)Cy-

wherein Cy is cycloalkyl, cycloalkenyl, cycloalkadienyl, heterocyclic, mono-or poly-unsaturated heterocyclic, aryl or heteroaryl, and

R^{b}′ is —(CR^{c}₂)₀₋₄—CHₘY₃₋ₘ,

wherein:

each Y is independently —OR^{d}, —COOR^{c}, —CF₃, —P(O)(OR^{c})₂, —OP(O)(OR^{c})₂, —NH—P(O)(OR^{c})₂, —NH—CH(CF₃)₂, each R^{c} is independently H or lower alkyl, each R^{d} is independently H, lower alkyl or alkylcarbonyl, and m=1 or 2.

Presently preferred compounds contemplated for use in the practice of the present invention are compounds comprising the following moieties:

A: —(CH₂)₂COOH; —(CH₂)₃COOH; —CH₂CH(COOH)₂; —(CH₂)₂CH(COOH)₂; —(CH₂)₂ CF₃; —(CH₂)₃CF₃; —CH₂CH(CF₃)₂; —(CH₂)₂CH(CF₃)₂; —CH₂COH(CF₃)₂; —(CH₂)₂COH(CF₃)₂; —CHCH₃OPO₃H₂; —CHCH₃NHPO₃H₂; —CHCH₃CH₂PO₃H₂; —CH₂OPO₃H₂; —(CH₂)₂OPO₃H₂; —CH₂NHPO₃H₂; —(CH₂)₂NHPO₃H₂; —(CH₂)₂PO₃H₂; or —(CH₂)₃PO₃H₂;

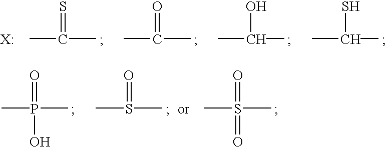

R: pyrrolyl, pyridyl, phenyl, or pentyl.

As used herein “alkyl” refers to straight or branched chain hydrocarbyl groups having up to 12 carbon atoms and “substituted alkyl” comprises alkyl groups further bearing one or more substitutions selected from hydroxy, alkoxy, (of a lower alkyl group), mecapto (of a lower alkyl group), cycloalkyl, substituted cycloalkyl, heterocyclic, substituted herocyclic, aryl, substituted aryl, heteroaryl, substituted heteroaryl, aryloxy, substituted aryloxy, halogen, trifluoromethyl, cyano, nitro, nitrone, amino, amido, —C(O)H, acyl, oxyacyl, carboxyl, carbamate, sulfonyl, sulfonamide, sulfuryl, and the like.

As used herein “alkenyl” refers to straight or branched chain hydrocarbyl groups having in the range of 2 up to 12 carbon atoms, additionally having one or more double bonds, and “substituted alkenyl” comprises alkenyl groups further bearing one or more substitutions as described above.

As used herein “alkynyl” refers to straight or branched chain hydrocarbyl groups having in the range of 2 up to 12 carbon atoms, additionally having one or more triple bonds, and “substituted alkynyl” comprises alkynyl groups further bearing one or more substitutions as described above.

As used herein, “cycloalkyl” refers to cyclic ring-containing groups containing in the range of about 3 to up to 13 carbon atoms, and “substituted cycloalkyl” refers to cycloalkyl groups further bearing one or more substituents as set forth above.

As used herein “heterocyclic” refers to cyclic (i.e. ring-containing) groups containing one or more heteroatoms (e.g., N, O, S, or the like) as part of the ring structure, and having in the range of 3 up to 14 carbon atoms and “substituted heterocyclic” refers to heterocyclic groups further bearing one or more substituents as set forth above.

As used herein “aryl” refers to aromatic groups having in the range of 6 up to 14 carbon atoms and “substituted aryl” refers to aryl groups further bearing one or more substituents as set forth above.

As used herein “heteroaryl” refers to aromatic groups containing one or more heteroatoms (ex. N, O, S, or the like) as part of their structure and having in the range of 3 up to 14 carbon atoms and “substituted heteroaryl” refers to heteroaryl groups further bearing one or more substituents as set forth above.

A further aspect of the invention encompasses methods of treatment using inhibitors of PPIase activity. Enzymes of the Pin1/parvulin class of PPIases are known to be essential for mitosis. Such enzymes have been identified in bacteria, fungi, insect and mammalian cells. Thus the compounds of the invention are useful for the treatment of a wide variety of disorders involving mitosis or cell proliferation.

Cell proliferative disorders contemplated for treatment using the invention compounds and methods disclosed herein include disorders characterized by unwanted, inappropriate or uncontrolled cell growth. Particular examples include cancer, fibrotic disorders, non-neoplastic growths such as benign prostatic hypertrophy, endometriosis, psoriasis, and the like. Cancers contemplated for treatment in accordance with the present invention include both solid tumors and hematopoeitic cancers such as leukemias and lymphomas.

Solid tumors that are treatable utilizing the invention compounds and methods include carcinomas, sarcomas, osteomas, fibrosarcomas, chondrosarcomas, and the like. Specific cancers contemplated for treatment include breast cancer, brain cancer, lung cancer (non-small cell and small cell), colon cancer, pancreatic cancer, prostate cancer, gastric cancer, bladder cancer, kidney cancer, head and neck cancer, and the like.

Fibrotic disorders are generally characterized by inappropriate overproliferation of non-cancerous fibroblasts. Examples include fibromyalgia, fibrosis (cystic, hepatic, idopathic pulmonary, pericardial, and the like), cardiac fibromas, fibromuscular hyperplasia, restenosis, atherosclerosis, fibromyositis, and the like.

Invention compounds are additionally useful in inhibiting mitosis in pathogenic organisms and are, therefore, useful for treating infectious diseases. Particular infectious diseases treatable by the methods disclosed herein include bacterial infections and fungal infections.

Bacterial infections contemplated for treatment using invention compounds and methods include infections caused by both gram-positive and gram-negative bacteria, including infections caused by Staphylococcus, Clostridium, Streptococcus, Enterococcus, Diplococcus, Hemophilus, Neisseria, Erysipelothricosis, Listeria, Bacillus, Salmonella, Shigella, Escherichia, Klebsiella, Enterobacter, Serratia, Proteus, Morganella, Providencia, Yersinia, Camphylobacter, Mycobacteria, and the like. Infection by such organisms causes a wide variety of disorders including pneumonia, diarrhea and dysentery, anthrax, rheumatic fever, toxic shock syndrome, mastoiditis, meningitis, gonorrhea, typhoid fever, gastroenteritis, brucellosis, cholera, bubonic plague, tetanus, tuberculosis, Lyme disease, and the like.

Fungal infections contemplated for treatment using invention compounds and methods include systemic fungal infections, dermatophytoses and fungal infections of the genito-unrinary tract. Systemic fungal infections include those caused by Histoplasma, Coccidioides, Cryptococcus, Blastocyces, Paracoccidioides, Candida, Aspergillus, Nocardia, Sporothrix, Rhizopus, Absidia, Mucor, Hormodendrum, Phialophora, Rhinosporidium, and the like. Dermatophyte infections include those caused by Microsporum, Trichophyton, Epidermophyton, Candida, Pityrosporum, and the like. Fungal disorders of the genito-urinary tract include infections caused by Candida, Cryptococcus, Aspergillus, Zygomycodoides, and the like. Infection by such organisms causes a wide variety of disorders such as ringworm, thrush, San Joaquin fever or Valley fever, Gilcrist's disease, and the like. These infections can be particularly serious, and even fatal, in patients with a depressed immune system such as organ transplant recipients and persons with acquired immunodefficiency syndrome (AIDS).

In a further aspect of the invention, invention compounds may be used as insecticides. The compounds of the invention prevent mitosis in insect cells, and thus can be used to control the growth and proliferation of a variety of insect pests. This aspect of the invention has important applications in agriculture, such as in the field, in the storage of agricultural products, and the like. Additionally, invention compounds are useful for controlling insect populations in places inhabited by man, such as homes, offices, and the like.

The particular invention compound(s) selected for therapeutic use as taught herein can be administered to a subject either alone or in a pharmaceutical composition where the compound(s) is mixed with suitable carriers or excipient(s). In treating a subject, a therapeutically effective dose of compound (i.e. active ingredient) is administered. A therapeutically effective dose refers to that amount of the active ingredient that produces amelioration of symptoms or a prolongation of survival of a subject.

Toxicity and therapeutic efficacy of a compound can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. Compounds which exhibit large therapeutic indices are preferred. The data obtained from these cell culture assays and animal studies can be used in formulating a range of dosages suitable for use in humans. The dosage of such compounds lies preferably within a range of circulating concentrations that include the ED₅₀ with little or no toxicity. The dosage may vary within this range depending upon a variety of factors, e.g., the dosage form employed, the route of administration utilized, the condition of the subject, and the like.

For any compound used in the method of the invention, the therapeutically effective dose can be estimated initially from cell culture assays by determining an IC₅₀ (i.e., the concentration of the test substance which achieves a half-maximal inhibition of PPIase activity). A dose can then be formulated in animal models to achieve a circulating plasma concentration range that includes the IC₅₀ as determined in cell culture. Such information can be used to more accurately determine useful doses in humans. Levels in plasma may be measured, for example, by HPLC. The exact formulation, route of administration and dosage can be chosen by the individual physician in view of the patient's condition. (See e.g. Fingl et al., 1975, in “The Pharmacological Basis of Therapeutics”, Ch. 1 pl).

It should be noted that the attending physician would know how to and when to terminate, interrupt, or adjust administration due to toxicity, to organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient. Typically, the dose will be between about 1-10 mg/kg of body weight. About 1 mg to about 50 mg will be administered to a child, and between about 25 mg and about 1000 mg will be administered to an adult. A program comparable to that discussed above may be used in veterinary medicine.

Depending on the specific conditions being treated, such agents may be formulated and administered systemically or locally. Techniques for formulation and administration may be found in “Remington's Pharmaceutical Sciences,” 1990, 18th ed., Mack Publishing Co., Easton, Pa. Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

For injection, compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

Use of pharmaceutically acceptable carriers to formulate the compounds herein disclosed into dosages suitable for systemic administration is within the scope of the invention. With proper choice of carrier and suitable manufacturing practice, the compositions of the present invention, in particular those formulated as solutions, may be administered parenterally, such as by intravenous injection. The compounds can be readily formulated using pharmaceutically acceptable carriers well known in the art into dosages suitable for oral administration. Such carriers enable the compounds of the invention to be formulated as tablets, pills, capsules, dragees, liquids, gels, syrups, slurries, suspensions and the like, for oral ingestion by a subject to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Delivery systems involving liposomes are discussed in International Patent Publication No. WO 91/02805 and International Patent Publication No. WO 91/19501, as well as U.S. Pat. No. 4,880,635 to Janoff et al. These publications and patents provide useful descriptions of techniques for liposome drug delivery and are incorporated by reference herein in their entirety.

Pharmaceutical compositions contemplated for use in the present invention include compositions wherein the active ingredients are contained in an amount effective to achieve the intended purpose. Determination of an effective amount is well within the capability of those skilled in the art, especially in light of the detailed disclosure provided herein.

In addition to the active ingredients, these pharmaceutical compositions may contain suitable pharmaceutically acceptable excipients and auxiliaries which facilitate processing of the active compounds into preparations which can be used pharmaceutically.

The pharmaceutical compositions of the present invention may be manufactured in a manner that is itself known, e.g., by means of conventional mixing, dissolving, granulating, dragee-making, levigating, emulsifying, encapsulating, entrapping, lyophilizing processes, or the like.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding the resulting mixture, and processing the mixture of granules, after adding suitable auxiliaries, if desired, to obtain tablets or dragee cores.

Suitable excipients are, in particular, fillers such as sugars, including lactose, sucrose, mannitol, sorbitol, and the like; cellulose preparations such as, for example, maize starch, wheat starch, rice starch, potato starch, gelatin, gum tragacanth, methyl cellulose, hydroxypropylmethylcellulose, sodium carboxymethylcellulose, polyvinylpyrrolidone (PVP), and the like, as well as mixtures of any two or more thereof. If desired, disintegrating agents may be added, such as cross-linked polyvinyl pyrrolidone, agar, alginic acid or a salt thereof such as sodium alginate, and the like.

Dragee cores are provided with suitable coatings. For this purpose, concentrated sugar solutions may be used, which may optionally contain gum arabic, talc, polyvinyl pyrrolidone, carbopol gel, polyethylene glycol, titanium dioxide, lacquer solutions, suitable organic solvents or solvent mixtures, and the like. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

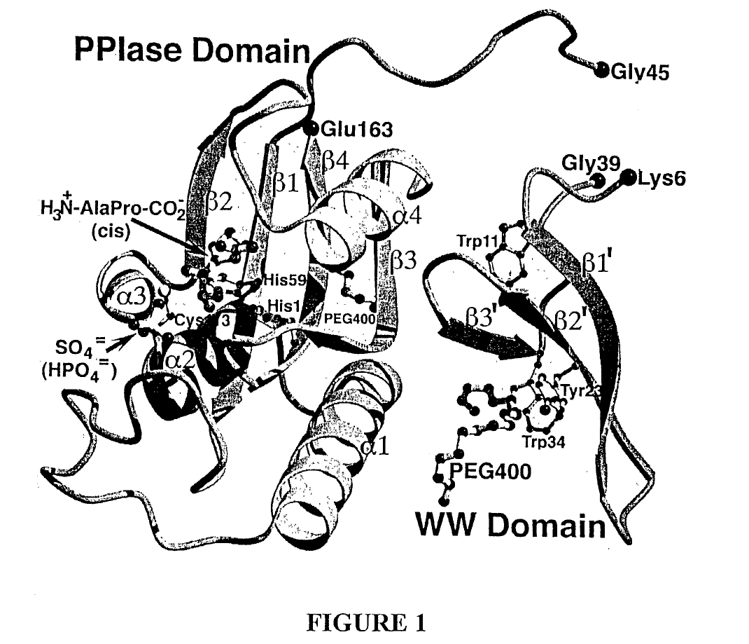

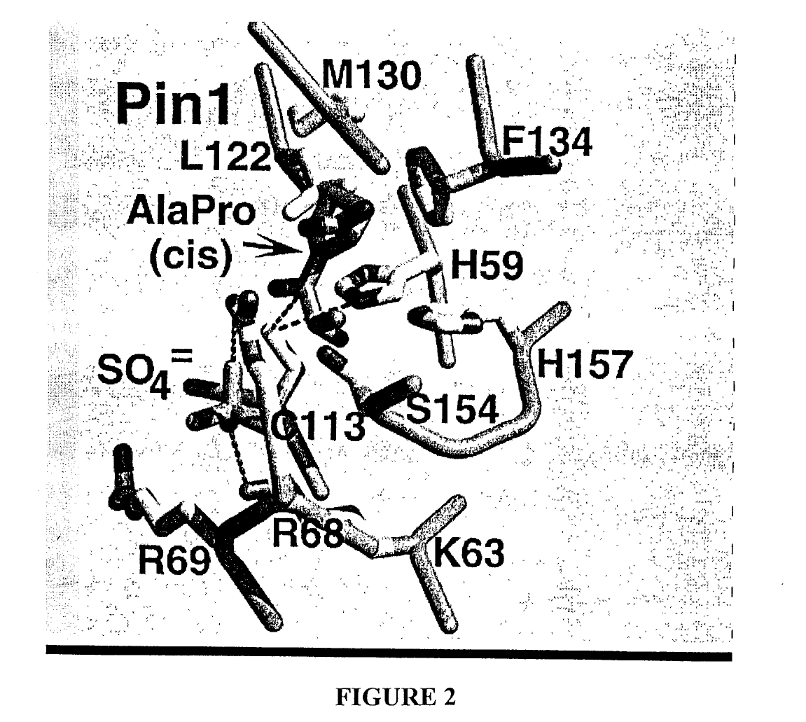

A further aspect of the invention comprises crystalline Pin1 (both alone and in a complex with the peptidyl substrate mimic AlaPro), the coordinates describing this crystal and methods of using them to design inhibitors of PPIase activity. The production of Pin1 crystals is described in detail in the Examples below. The resulting Pin1 crystals contain one Pin1 molecule per asymmetric unit and belong to space group P4₃2₁2. Two crystal forms predominate; I: a=b=47.6 Å, c=134.9 Å, α=β=γ=90°; and II: a=b=49.0 Å, c=137.8 Å, α=β=γ=90°. The crystal coordinates are shown in FIGS. 1 and 2.

Methods of using crystal structure data to design inhibitors of enzyme activity are known in the art. Thus, the crystal structure data provided herein can be used in the design of new or improved enzymatic inhibitors. For example, the Pin1 coordinates can be superimposed onto other available coordinates of similar enzymes which have inhibitors bound to them to give an approximation of the way these and related inhibitors might bind to Pin1. Alternatively, computer programs employed in the practice of rational drug design can be used to identify compounds that reproduce interaction characteristics similar to those found between Pin1 and the co-crystalized substrate mimic. Furthermore, detailed knowledge of the nature of binding site interactions allows for the modification of compounds to alter or improve solubility, pharmacokinetics, etc. without affecting binding activity.

Computer programs are widely available that are capable of carrying out the activities necessary to design compounds using the crystal structure information provided herein. Examples include, but are not limited to, the computer programs listed below:

Catalyst Databases™—an information retrieval program accessing chemical databases such as BioByte Master File, Derwent WDI and ACD;

Catalyst/HYPO™—generates models of compounds and hypotheses to explain variations of activity with the structure of drug candidates;

Ludi™—fits molecules into the active site of a protein by identifying and matching complementary polar and hydrophobic groups;

Leapfrog™—“grows” new ligands using a genetic algorithm with parameters under the control of the user.

In accordance with the present invention, the molecular features important in Pin1:substrate interaction and catalytic activity have been determined and these features have been incorporated into the compounds disclosed herein. Structural analysis revealed that a hydrophobic pocket composed of Pin1 residues Phe-134, Met-130 and Leu-122 forms the binding site for the hydrophobic cyclic side chain of the substrate proline, and that the peptidyl-prolyl bond undergoing catalyzed isomerization is surrounded by the side chains of Pin1 residues Cys-113, His-59, His-157 and Ser-154. These latter residues are symmetrically distributed around the bond rotation axis and create conformation-specific interactions with the substrate during isomerization. These interactions, among other things, help form and stabilize a tetrahedral intermediate as rotation proceeds around the carbonyl carbon of the peptidyl bond.

Finally, Pin1 residues Lys-63, Arg-68 and Arg-69 form a basic cluster at the entrance to the enzyme's active site. The spatial proximity of this cluster in the active site to the bound dipeptide indicates that this anionic recognition site confers preferential binding to substrates with an acidic residue N-terminal to the proline. Indeed, a glutamate, phosphoserine or phosphothreonine side chain modeled on the Ala of the AlaPro dipeptide superimposes its respective anionic group on the bound sulfate ion in the complex crystal structure.

These enzyme:substrate interactions were employed in selecting design criteria for PPIase inhibitors. Thus compounds of the invention have been designed to include four general features: 1) hydrophobicity similar to that of proline, 2) maintenance of enzymatic specificity by including charged acidic groups or trifluoromethyl groups in place of the preferred phosphoserine/phosphothreonine, 3) mimicking of the tetrahedral state assumed by the prolyl ring nitrogen during isomerization and 4) maintenance of rotatability of the carbonyl moiety of the rotating peptide bond.

The rotatability and geometry of the prolyl peptide bond has been mimicked in the compounds of the invention in several ways. First, the substitution of a secondary alcohol for the carbonyl introduces a tetrahedral center at this position and positions the compound's hydroxyl groups for efficient hydrogen bonding to either His-59 or His-157 in the enzyme active site. The second approach takes advantage of the presumed reactivity of Cys-113 and the proton donating capability of His-157. Unlike the restricted rotation of the peptide bond, introduction of a ketone moiety at this position allows free rotation of the ketone moiety in the enzyme active site. Given the positioning of the Cys-113 and His-157 side chains near the ketone carbon, reversible hemithioketal formation is likely to occur, leading to stable inhibition of enzyme activity. The third approach employs tetrahedral mimics such as sulfonamide, phosphonate, and phosphonamidate analogs in place of the carbonyl moiety.

The invention will now be described in greater detail by reference to the following non-limiting Examples.

### EXAMPLE 1

### Methods of Synthesis

Those of skill in the art recognize that a variety of synthetic techniques can be employed to prepare invention compounds. For example, ethers can be made by reduction of esters using suitable reducing agents, e.g., boron trifluoride-trietherate or lithium aluminum hydride; alternatively, ethers can be made by reduction of thiocarbonyl esters with Raney nickel; esters can be made by condensation of acyl halides with alcohols; aldehydes can be prepared by oxidation of alcohols using suitable oxidizing agents, e.g., Cu(I)Cl plus 1,10-phenanthroline; ketones can be prepared by reaction of Weinreb amides with a suitable Grignard reagent; and the like.

As additional examples of synthetic Trifluoromethyl-substituted compounds can be readily prepared by condensation of a fluorinated ketone (e.g., perfluoroacetone) with an amine, followed by reduction of the resulting intermediate with a suitable reducing agent (e.g., sodium borohydride).

Phosphorylated compounds can readily be prepared by treatment of an alcohol or an amine with (tBuO)₂PN(iPr)₂ plus tetrazole, then oxidizing the resulting phosphate ester with a suitable oxidizing agent, e.g., tBu-OOH, then finally hydrolyzing the resulting material with a suitable acid, e.g., trifluoroacetic acid.

### EXAMPLE 2

### In vitro Assays for Inhibitors of Pin1 Activity

This example describes three in vitro assays useful for identification of inhibitors of the activity of enzymes of the Pin1/parvulin class.

The first assay measures whether a test substance can enhance or inhibit the ability of Pin1 to catalyze the isomerization of a tetrapeptide substrate using a protocol modified from Heitman, et al (METHODS: A Comparison to Methods in Enzymology 5:176-187, 1993) and Kofron, et al (Biochemistry 30:6127-6134, 1991).

Briefly, purified Pin1 (see Example 4 below) is diluted into assay buffer (50 mM succinic acid/bis-Tris propane, pH 7.5, 100 mM NaCl) immediately prior to performing the assay. A 900 μl cocktail containing Pin1 (36 mM final concentration) and 20 μM substrate (succinyl-AlaGluProPhe-(p)-nitroanilide, 15 μM (Bachem, Inc) Ala-phosphorylated Ser-Pro Phe-(p)-nitroanilide) and a test substance diluted in a suitable diluent such as water, phosphate buffered saline (PBS) or dimethylsulfoxide (DMSO) is equilibrated in a spectrophotometer at 3.6° C. A chilled chymotrypsin (Sigma) solution (100 μl, 1 mM in water) is added, mixed for 5 seconds and the absorbance of p-nitroaniline at 395 nm measured over a period of 10 minutes. The relative absorbance curve of assay mixtures containing a test substance is compared to controls having no test substance and controls having an inhibitory amount (100-500 μM) of organic phosphate.

The second assay measures the ability of a test substance to regulate mitosis in yeast. This assay is described in Lu, et al, Nature 380:544-547, 1996. Briefly, a haploid ess1⁻ yeast strain is genetically engineered to express Pin1 under control of the Gall promoter. This strain grows normally in galactose containing media (inducing media) but does not grow in glucose containing media (repressing media), demonstrating the absolute requirement for a functional Pin1 for continued growth. Test substance diluted in a suitable diluent is added to cells grown in inducing media at varying concentrations. Control cells receive no test substance. After some period of time, the total amount of cells is measured. A reduction in cell number compared to control cells indicates that the test substance may be a Pin1 inhibitor.

The third assay measures the ability of a test substance to inhibit the growth of transformed cells in soft agar. The assay uses transformed mammalian cells and is, therefore, predictive of the ability of a substance to be useful as a treatment for cell proliferative disorders in mammals such as, for example, cancer. It is recognized by those skilled in the art that similar well known assays exist for predicting the ability of a substance to be useful as an antibacterial agent or an antifungal agent.

Briefly, transformed cells, such as ovarian cancer cell line SKOV-3 (ATCC HTB77), are grown until confluent (Dulbecco's Modified Eagle's Medium (DMEM), 20% FBS, 2 mM Na-pyruvate, 4 mM glutamine, 20 mM HEPES, non-essential amino acids), trypsinized, then washed in PBS and resuspended in DMEM 10% FBS, 1 mM Na-pyruvate, 2 mM glutamine, 10 mM HEPES, and non-essential amino acids (assay medium). Cells are then suspended in assay medium plus 0.8% SeaPlaque Agarose and test substance diluted to varying concentrations in an appropriate diluent. This mixture is placed in a petri dish or wells of a multi-well plastic plate pre-plated with a base layer of assay medium plus 0.8% agarose. The cells are incubated for 2-3 weeks in a 100% humidified, 10% CO₂ incubator after which time colonies ≧60 microns in size are counted. A reduction in the number of colonies, compared to cells that received no test substance is indicative of a Pin1 inhibitor.

### EXAMPLE 3

### In vivo Assays for Inhibitors of Pin1 Activity

The following example describes an in vivo assay for evaluating the ability of a test substance to inhibit Pin1 activity. Those of skill in the art will recognize that other in vivo assays appropriate to a particular disorder can be used for further evaluation.

The ability of tumors to grow as xenografts in athymic mice (example Balb/c, nu/nu) provides a useful in vivo model for studying the biological response to therapies for human tumors. A variety of tumor types have been successfully xenotransplanted into athymic mice (see Rygaard and Povlsen Acta Pathol. Microbial. Scan. 77:758-760, 1969; Ward, et al, Int J Cancer 49:616-623, 1991, for example). Briefly, tumor cells are implanted subcutaneously into the hindflank of five- to six-week old female Balb/c nu/nu athymic mice. The test substance in an appropriate vehicle is administered to the animal in periodic doses (orally, intravenously, intraperitoneally, etc.). Growth of the tumor cells is measured over time in comparison with animals not receiving any test substance, or receiving vehicle alone. A reduction in tumor size as compared to control animals is indicative of substances useful as therapeutics for treating cell proliferative disorders.

### EXAMPLE 4

### Preparation and Use of Crystalline Pin1

The following example describes the formation of crystalline Pin1 and illustrates methods for using the information obtained to design inhibitors of the Pin1/parvulin class of enzymes. (See also Ranganathan, et al, Cell 89:875-886, 1997, incorporated by reference herein in its entirety.)

N-terminally His₆-tagged Pin1 (Lu, et al, 1996, supra) was expressed at 22° C. in E. coli strain BL21(DE3) following induction at an optical density of 1.2 (600 nm) with 0.4 mM IPTG for 4 hr in terrific broth. Cells were centrifuged into a pellet and resuspended in 25 mM Tris-HCl (pH 8.0), 500 mM NaCl, 10 mM imidazole, 10 mM β-mercaptoethanol, and 1% (v/v) Tween 20 on ice (sonication buffer). Following sonication at 4° C., the soluble supernatant was loaded onto an Ni-NTA (Quiagen) column and washed with sonication buffer minus Tween 20. His₆-Pin1 was eluted with 15 bed volumes of sonication buffer minus Tween 20 and supplemented with 250 mM imidazole. Eluted His₆-Pin1 was digested with thrombin (Sigma) during dialysis for 12 hr at 4° C. against 50 mM Tris-HCl (pH 8.0), 150 mM NaCl, 5 mM MgCl₂, 2.5 mM CaCl₂ 1 mM DTT, and 10% (v/v) glycerol, depleted of thrombin with a benzamidine-Sepharose column (Pharmacia), and fractionated by gel filtration on a Superdex 75 16/60 column (Pharmacia) equilibrated in 10 mM HEPES-Na⁺ (pH 7.5), 100 mM NaCl, and 1 mM DTT. The Pin1-containing fractions were concentrated to 20 mg/ml with a Centricon-10 (Amicon) and stored at −80° C.

Crystals were grown in hanging drops at 4° C. by mixing 5 μl of concentrated Pin1 (20 mg/ml) with 5 μl of a reservoir solution consisting of 2.00-2.50 M ammonium sulfate, 100 mM HEPES-Na⁺ (pH 7.5), 1% (v/v) PEG400 (Sigma), and 1 mM dithiothreitol (DTT) (stabilizer). The stabilized crystals were frozen in a stream of 100° K nitrogen gas. The crystals contain one Pin1 molecule per asymmetric unit and belong to space group P4₃2₁2. Two crystal forms predominate; I: a=b=47.6 Å, c=134.9 Å, α=β=γ=90°; and II: a=b=49.0 Å, c=137.8 Å, α=β=γ=90°. The complex of Pin1 with AlaPro dipeptide (AP II) was obtained by soaking a type II crystal in 40% (v/v) PEG400, 50 mM HEPES-Na⁺ (pH 7.5), 50 mM ⁺H₃N-AlaPro-COO⁻ (Sigma) for 48 hours at 4° C. A single site TAMM (tetrakis(acetoxymercuri)methane, Strem Chemical, Inc) derivative was obtained by soaking Pin1 crystals for 12 hours at 4° C. in the stabilizer (minus DTT) saturated with TAMM. The five-site PIP (di-μ-idodobis(ethylenediamine)-diplatinum(II)nitrate, Strem Chemical Inc.) derivative was obtained by soaking Pin1 crystals for 48 hours at 4° C. in the stabilizer (minus DTT) supplemented with 10 mM PIP.

Native (Nat I, 2.05 Å) and derivative data (2.5 Å) for crystal form I were collected on a MacScience imaging plate detector, DIP2020k (MacScience Corp.) using double focusing Pt/Ni-coated mirrors and Cu Kα X-rays from a MacScience M18XHF generator operating at 4.5 kW (50 kV×90 mA). Data for Pin1 complexed with AlaPro dipeptide for crystal form II (AP II) were collected at the Stanford Synchrotron Radiation Laboratory, beamline 7-1 (λ=1.08 Å) on a MAR imaging plate system. Data were processed with DENZO (Otwinowski, in Data Collection and Processing, pp 55-62, 1993) and scaled with SCALEPACK (Otwinowski, supra). A single mercury binding site for the TAMM derivative was located on the isomorphous difference Patterson map and refined with ML-PHARE. Solvent flattening, histogram matching, and Sayre's equation were employed to improve and extend phases to 2.05 Å resolution using DM (Cowtan, Newsletter on Protein Crystallography 31:34-38, 1994). Model building was conducted with 0 (Jones, et al, Acta Crystallography A47:110-119, 1991), and the structures were refined with X-PLOR (Brunger, X-PLOR Version 3.1: A System for X-Ray Crystallography and NMR (New Haven, Conn.: Dept. of Mol. Biophysics and Biochem. and Howard Hughes Med. Inst., Yale University, 1992). The initial native model (Nat I, residues 6-39, 45-163) was refined following partial solvent modeling (60 water molecules added) using all the data (no sigma cutoff) between 6.0 Å and 2.05 Å resolution. Subsequently, the Pin1:AlaPro complex (AP II) was solved using the Nat I model as a starting point for rigid body refinement in X-PLOR. Following positional and simulated annealing refinement, 208 water molecules, 2 PEGs)) molecules, 1 sulfate ion, and 1 cis AlaPro dipeptide were modeled and refined with X-PLOR using all the data between 6.0 Å and 1.35 Å resolution. The crystallographic data are summarized in Table 1 below.

TABLE I
Summary of Crystallographic Data

| | | | | | | | |
|---|---|---|---|---|---|---|---|
| Data | | | Reflections | | | | |
| Set | | Resolution | Measured | Completeness | Rsym %^{a} | <I/σ> | |
| Sites | Source | Limit(Å) | (Unique) | All/Outer Shell | All/Outer Shell | All/Outer Shell | Heavy Atom |

Data Collection Statistics

| | | | | | | | |
|---|---|---|---|---|---|---|---|
| Nat I | Salk | 2.05 | 11139 | 99.7/99.9 | 6.7/45.3 | 21/4 | — |
| AP II | SSRL 7-1 | 1.35 | 33672 | 95.5/69.0 | 5.3/59.2 | 18/2 | — |
| TAMM I | Salk | 2.5 | 9751^{b} | 87.8/87.7 | 5.8/22.2 | 14/5 | “1” |
| PIP I | Salk | 2.5 | 10986^{b} | 99.4/98.5 | 6.6/35.3 | 19/4 | “5” |

Multiple Isomorphous Replacement with Anomalous Scattering Statistics

| | | | | | | | | | |
|---|---|---|---|---|---|---|---|---|---|
| Resolution (Å) | 20-10.54 | 7.07 | 5.42 | 4.39 | 3.70 | 3.19 | 2.81 | 2.50 | Overall |
| Phasing Power^{c} | | | | | | | | | |
| TAMM Isomorphous | 1.52 | 1.57 | 2.26 | 1.85 | 1.35 | 1.26 | 1.43 | 1.35 | 1.46 |
| TAMM Anomalous | 0.84 | 0.90 | 0.88 | 0.71 | 0.50 | 0.42 | — | — | 0.63 |
| PIP Isomorphous | 1.46 | 1.71 | 2.10 | 1.76 | 1.44 | 1.51 | 1.70 | 1.66 | 1.64 |
| PIP Anomalous | 1.00 | 0.92 | 1.03 | 0.84 | 0.66 | 0.50 | 0.39 | — | 0.63 |
| Mean figure of | 0.66 | 0.67 | 0.73 | 0.69 | 0.59 | 0.53 | 0.51 | 0.45 | 0.54 |
| merit^{d} | | | | | | | | | |

| Data | Resolution | | Free R | Unique Reflections |
|---|---|---|---|---|
| Set | Range (Å) | R factor^{e} | factor^{e} | working/test^{e} |
| Refinement Statistics | | | | |
| NatI | 6.00-2.05 | 25.6 | 31.2 | 10090/505 |
| AP II | 6.00-1.35 | 22.3 | 26.6 | 31532/1678 |

| | | | |
|---|---|---|---|
| 17.8 main chain (AP II) | 1.0 main chain (AP II) | 0.005 (Nat I) | 1.39 (Nat I) |
| 22.8 side chain (AP II) | 2.0 side chain (AP II) | 0.008 (AP II) | 1.78 (AP II) |
| Average B-factor (Å²) | B-factor RMSD (Å²) | Bond length RMSD (Å) | Bond angle RMSD (°) |

^{a}Rsym = 3ₕ|Iₕ − <Iₕ> |/3ₕIₕ where <Iₕ> is the average intensity of reflection h for its symmetry and Friedel equivalents.
^{C}Phasing power = 3 |Fₕ^{c}|/3 |∥Fₚ^{o}|exp(iφ_{c}) + Fₕ^{c}|−|Fₚₕ^{o}∥, where Fₕ^{c} = heavy-atom structure factor, and |Fₚ^{o}| and |Fₚₕ^{o}| are observed amplitudes for the protein and heavy-atom derivatives respectively, and φc is the experimental phase.
^{b}Friedel pairs were not merged. Therefore, the nwnber of unique and measured reflections reflects this non-equivalence.
^{d}Figure of merit = | P(φ)exp(iφ)dφ/| P(φ)dφ where P is the probability distribution of φ, the phase angle.
^{e}R factor = 3ₕ(|Fₒ(h) − F_{c}(h)|)/3ₕFₒ(h), where Fₒ(h) and F_{c}(h) are the observed and calculated structure factor amplitudes for reflection h, respectively. The Free R factor is calculated in an analogous manner for 5% of the data that has never been used for refinement. The R factor is calculated with the remaining 95% of the measured data. Both values are calculated with no sigma cutoff.

To define potential protein-protein interaction surfaces, the degree of conserved, solvent-exposed hydrophobicity for the ith residue was quantitatively assessed as a parameter ai, defined as:

ai=(conservation index)ᵢ(fractional solvent accessibility)ᵢ(hydrophobicity index)ᵢ.

The highlights solvent exposed hydrophobic patches that are often maintained due to functional necessity as protein-protein interaction surfaces. A fractional conservation index was assigned for each Pin1 residue from the alignment with its functional homologue from yeast, Ess1, where this index was taken as 0 for not conserved, 0.5 for chemically conserved, and 1 for identical. The solvent-accessible surface area for each residue was calculated using the CCP4 program RESAREA (Collaborative Computational Project, Number 4 (1994) Acta Cryst. D5°, 760-763. “The CCP4 Suite: Programs for Protein Crystallography”) and was divided by the total surface area to give the fractional solvent accessibility. The hydrophobicity index is the ratio of the probability of finding a given residue in the interior to that on the surface (P/P₀) and is calculated as e^{-ΔG/RT} where the free energy is normalized such that ΔG_{Gly}=0. (Miller et al., J. Mol Biol. 196:641-656, 1987; Creighton, Protein Structures and Molecular Properties, NY: W. H. Freemean and Co. 1993). Values of a₁ were mapped onto a color scale and displayed on a molecular surface representation of Pin1 using GRASP (Nicholls et al., Proteins 11:281-296, 1991).

To further define the key binding-site interaction, site-directed mutations were introduced using PCR-based techniques and verified by automated sequencing. The corresponding proteins were expressed and purified as described above. PPIase activity was measured by a protocol modified from Heitman et al, METHODS: A Companion to Methods in Enzymology 5:176-187, 1993 and Kofron et al, (1991) supra. Purified Pin1 was diluted into assay buffer (50 mM succinic acid/bis-Tris propane at indicated pH values, 100 mM NaCl) immediately prior to kinetic measurements. A 900 μI cocktail containing Pin1 and 15-20 μM substrate was equilibrated in the spectrophotometer at 3.6° C. A chilled chymotrypsin (Sigma) solution (100 μI, 1 mM in water) was added, mixed for 5 s, and the absorbance of p-nitroaniline (at 395 nM) was followed every 6 s for 2-10 min. Total absorbance was normalized to zero immediately prior to data acquisition, and substrate concentration was adjusted to remain within the linear range of the instrument. Data were analyzed off-line using a combination of Excel 7.0 (Microsoft Corp.) and Origin 4.1 (Microcal Software). The PPIase rate-limited portion of each curve was well fit to a single exponential decay function of the form 1-ae_{bt} where a and b were free 2 parameters. Goodness of fit was assessed by standard χ² analyses. For inorganic phosphate inhibition assays, the indicated concentration of sodium phosphate buffer (pH 7.0) was added from a 1 M stock. Substrate peptides were from Bachem Inc.

The data obtained from these experiments were used to construct a 3-dimentional model of Pin1:substrate binding and to elucidate the interactions key for Pin1 specificity and activity. This knowledge was, in turn, used in the design of inhibitors.

While the foregoing has been with reference to particular embodiments of the invention, it will be appreciated by those skilled in the art that changes in these embodiments may be made without departing from the principles and spirit of the invention, the scope of which is defined by the appended claims.

1. A method for inhibiting the activity of a peptidyl-prolyl cis-trans isomerase, said method comprising contacting the isomerase with an effective amount of a compound having the structure:
A-X-R (i)
wherein:
A is a radical which mimics the steric and electronic properties of a phosphoserine and/or phosphothreonine residue,
X is a spacer, and
R is cycloalkyl, substituted cycloalkyl, heterocyclic, substituted heterocyclic, aryl, substituted aryl, heteroaryl, or substituted heteroaryl, wherein R is at least as hydrophobic as a pyrrolidine ring substituted with a hydrophilic moiety,
wherein A is selected from radical II, radical III or radical IV,
wherein radical II has the structure: 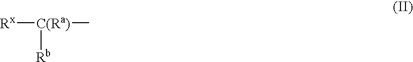
wherein:
R^{x} is an organic radical having a molecular weight no greater than about 250,
R^{a} is H, halo or lower alkyl, and
R^{b} is —(CR^{c}₂)₁₋₄—CHₘY₃₋ₘ, wherein:
each Y is independently —OR^{d, —COOR}^{c}, —CF₃, —P(O)(OR^{c})₂, —OP(O)(OR^{c})₂, —NH—P(O)(OR^{c})₂, —NH—CH(CF₃)₂,
each R^{c} is independently H or lower alkyl, and each R^{d} is independently H, lower alkyl or alkylcarbonyl, and m=1 or 2;
wherein radical III has the structure:
R^{z}—NH— (III)
wherein R^{z} is alkyl, substituted alkyl cycloalkyl, substituted cycloalkyl, cycloalkenyl, substituted cycloalkenyl, cycloalkadienyl, substituted cycloalkadienyl, heterocyclic, substituted heterocyclic, mono- or poly-unsaturated heterocyclic or substituted_mono- or poly-unsaturated heterocyclic, aryl, substituted aryl, heteroaryl, or substituted heteroaryl; and
wherein radical IV has the structure:
R^{b}-substituted Cy(het)- (IV)
wherein:
R^{b} is —(CR^{c}₂)1-4—CHₘY₃₋ₘ, wherein:
each Y is independently —OR^{d}, —COOR^{c}, —CF₃, —P(O)(OR^{c})₂, —OP(O)(OR^{c})₂, —NH—P(O)(OR^{c})₂, —NH—CH(CF₃)₂,
each R^{c} is independently H or lower alkyl,
each R^{d} is independently H, lower alkyl or alkylcarbonyl, and m=1 or 2, and
Cy(het) is a 5, 6 or 7-membered heterocyclic ring wherein the heterocyclic atom thereof is linked to X of structure I.

2. A method according to claim 1 wherein the peptidyl-prolyl cis-trans isomerase is of the parvulin/Pin1 class.

3. A method according to claim 1 wherein the peptidyl-prolyl cis-trans isomerase regulates part of the cell cycle.

4. A method according to claim 3 wherein the part of the cell cycle being regulated is mitosis.

5. A method according to claim 1 wherein the peptidyl-prolyl cis-trans isomerase is mammalian.

6. A method according to claim 1 wherein the peptidyl-prolyl cis-trans isomerase is Pin1.

7. A method according to claim 1 wherein the compound of structure I mimics the tetrahedral intermediate involved in Pin1-mediated peptidyl-prolyl isomerization.

8. A method according to claim 1 wherein A of structure I is radical II.

9. A method according to claim 1 wherein R^{x} of radical II is alkyl, substituted alkyl, cycloalkyl or substituted cycloalkyl, cycloalkenyl or substituted cycloalkenyl, cycloalkadienyl or substituted cycloalkadienyl, heterocyclic or substituted heterocyclic, mono- or poly-unsaturated heterocyclic or substituted mono- or poly-unsaturated heterocyclic, aryl or substituted aryl, heteroaryl or substituted heteroaryl, or:
R^{y}-Q-
wherein:
R^{y} is alkyl, substituted alkyl, cycloalkyl, substituted cycloalkyl, cycloalkenyl, substituted cycloalkenyl, cycloalkadienyl, substituted cycloalkadienyl, heterocyclic, substituted heterocyclic, mono- or poly-unsaturated heterocyclic or substituted mono- or poly-unsaturated heterocyclic, aryl, substituted aryl, heteroaryl, or substituted heteroaryl, and
Q is —C(O)—NH—, —C(O)—O—, —C(O)—, or —O—.

10. A method according to claim 9 wherein R^{x} is an amino acid residue.

11. A method according to claim 10 wherein said amino acid residue is a leucinyl moiety, or a prolyl moiety.

12. A method according to claim 1 wherein R^{x} of radical II is

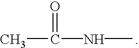

13. A method according to claim 1 wherein A of structure I is radical III.

14. A method according to claim 1 wherein R^{z} of radical III is 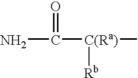
wherein:
R^{a} is H, halo or lower alkyl, and
R^{b} is —(CR^{c}₂)₁₋₄—CHₘY₃₋ₘ,
wherein:
each Y is independently —OR^{d}, —COOR^{c}, —CF₃, —P(O)(OR^{c})₂, —OP(O)(OR^{c})₂, —NH—P(O)(OR^{c})₂, or —NH—CH(CF₃)₂,
each R^{c} is independently H or lower alkyl,
each R^{d} is independently H, lower alkyl or alkylcarbonyl, and m=1 or 2.

15. A method according to claim 1 wherein R^{z} of radical III is
(R^{b}′)Cy-
wherein:
Cy is cycloalkyl, cycloalkenyl, cycloalkadienyl, heterocyclic, mono- or poly-unsaturated heterocyclic, aryl or heteroaryl, and
R^{b}′ is —(CR^{c}₂)₀₋₄—CHₘY₃₋ₘ,
wherein:
each Y is independently —OR^{d}, —COOR^{c}, —CF₃, —P(O)(OR^{c})₂, —OP(O)(OR^{c})₂, —NH—P(O)(OR^{c})₂, or —NH—CH(CF₃)₂,
each R^{c} is independently H or lower alkyl,
each R^{d} is independently H, lower alkyl or alkylcarbonyl, and m=1 or 2.

16. A method according to claim 1 wherein A of structure I is radical IV.

17. A method according to claim 1 wherein X is selected from:

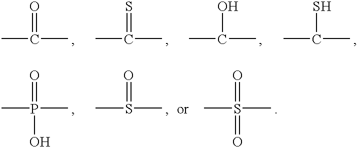

18. A method according to claim 1 wherein R is cycloalkyl or substituted cycloalkyl.

19. A method according to claim 18 wherein R is a 5-7 membered ring.

20. A method according to claim 1 wherein the peptidyl-propyl cis-trans isomerase is in a cell-free environment.

21. A method according to claim 1 wherein the peptidyl-propyl cis-trans isomerase is in a cell.

22. A method according to claim 21 wherein the contact occurs in vitro.

23. A method according to claim 21 wherein the contacting occurs in vivo.

24. A method according to claim 21 wherein the contacting modulates growth of the cell.

25. A method according to claim 24 wherein the cell is an insect cell, a fungal cell, a mammalian cell or a bacterial cell.

26. A method according to claim 1 wherein said compound is:

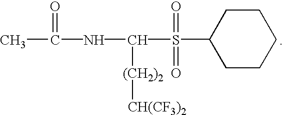

27. A method according to claim 1 wherein R is heterocyclic or substituted heterocyclic.

28 A method according to claim 27 wherein R is a 5-7 membered ring.

29 A method according to claim 1 wherein R is aryl or substituted aryl.

30. A method according to claim 29 wherein R is a 5-7 membered ring.

31. A method according to claim 1 wherein R is heteroaryl, or substituted heteroaryl.

32. A method according to claim 31 wherein R is a 5-7 membered ring.
